(19) United States
(12) Patent Application Publication
(10) Pub. No.: US 2011/0086875 A1
(43) Pub. Date: Apr. 14, 2011

(54) Powdered Medicament for Nasal Delivery of Ascorbic Acid for Reducing Apomorphine Induced Toxicity to Ciliated Tissue

(75) Inventors: Peter Alan Lambert, Victoria (AU); Franciscus Merkus, Kasterlee (BE)

(73) Assignee: BRITANNIA PHARMACEUTICALS LIMITED, Redhill, Surrey (GB)

(21) Appl. No.: 12/672,188

(22) PCT Filed: Aug. 4, 2008
(86) PCT No.: PCT/GB2008/002648

§ 371 Date: Dec. 6, 2010

(30) Foreign Application Priority Data
Aug. 6, 2007 (GB) 0715285.3

(51) Int. Cl. A61K 31/435; A61K 31/34; A61P 25/16; A61P 15/10

(52) U.S. Cl. 514/284; 514/474

(57) ABSTRACT

Use of ascorbic acid in the manufacture of a powdered medicament for nasal delivery, said medicament comprising an active agent such as apomorphine that exhibits toxicity to ciliated tissue, for ameliorating said toxicity.

The present invention relates to powdered medicaments for nasal delivery, and has particular reference to powdered medicaments comprising active agents that demonstrate a degree of toxicity to ciliated tissue. The powdered medicaments of the present invention are adapted to ameliorate such toxicity.

U.S. Pat. No. 5,756,483 (Merkus), the contents of which are incorporated herein by reference, discloses pharmaceutical compositions for intranasal administration of apomorphine, said compositions comprising apomorphine and/or apomorphine salts and cyclodextrin and/or other saccharides and/or sugar alcohols. Apomorphine is a potent dopamine agonist and is used as an adjunctive medication in the treatment of Parkinson's disease complicated by motor fluctuations, and is also indicated for the treatment of sexual dysfunction. Such nasal compositions can be made by mixing the active agent and the excipient, both possessing the desired particle size. Other methods to make a suitable powder formulation can be selected. Firstly, a solution of the active agent and the cyclodextrin and/or the other saccharides and/or sugar alcohols made, followed by precipitation, filtration and pulverisation. It is also possible to remove the solvent by freeze-drying, followed by pulverisation of the powder in the desired particle size by using conventional techniques, known from the pharmaceutical literature. According to U.S. Pat. No. 5,756,483, many other excipients, known from the pharmaceutical literature, can be added, such as preservatives, surfactants, co-solvents, adhesives, antioxidants, buffers, viscosity enhancing agents, and agents to adjust the pH or the osmolarity.

WO-A-2004/075824, the contents of which are also incorporated herein by reference, discloses formulations suitable for nasal delivery of pharmacologically and therapeutically active agents for systemic activity, in particular nasal powders containing drugs such as apomorphine and dihydroergotamine and their salts. According to WO-A-2004/075824, powder formulations of active materials and excipients of suitable particle size for nasal delivery can be directly obtained by freeze-drying, without the need for milling and without containing significant amounts of finings having a particle size of less than about 10 μm, by the selection of components with a specific crystalline/amorphous balance. Such powders retain free-flowing properties on storage, are physically and chemically stable and are readily soluble. According to WO-A-2004/075824, such powdered pharmaceutical formulations for nasal delivery comprise a freeze-dried blend of active material(s) and excipients(s) containing 0.5-50% wt. of the active material(s), and 50-99.5% wt. of excipient(s), in which at least 0.1% wt. of the blend is in an amorphous state.

The present inventors have observed that whilst the powder formulations of U.S. Pat. No. 5,756,483 and WO-A-2004/075824 perform generally satisfactory when administered to patients in need thereof, apomorphine demonstrates undesirable toxicity to ciliated tissue of the kind found in the nasal cavity (or nasal fossa). In particular, apomorphine has been observed to cause the arrest of cilia beat movement when applied in solution to a section of ciliated tissue taken from the trachea of a chick embryo maintained in Locke-Ringer solution. As those skilled in the art will appreciate, this is undesirable, since cilia beat movement is necessary to ensure the proper clearance of particles from the nasal cavity. Particles remaining uncleared in the nasal cavity, especially particles containing active drug substances, may give rise to undesirable nasal irritation as well as other, more serious conditions or symptoms.

Accordingly, it is an object to the present invention to provide an improved powdered medicament for nasal delivery comprising an active agent that demonstrates toxicity to ciliated tissue, especially the respiratory epithelium, which medicament is adapted to ameliorate such toxicity.

According to one aspect of the present invention therefore there is provided the use of ascorbic acid in the manufacture of a powdered medicament for nasal delivery, said medicament comprising an active agent that exhibits toxicity to ciliated tissue, for ameliorating said toxicity.

According to another aspect of the present invention there is provided a nasally-administered, powdered medicament for ameliorating the toxicity to ciliated tissue of a co-administered active agent that exhibits such toxicity, said medicament comprising said active agent and ascorbic acid.

By “toxic” herein is meant that the active agent has an adverse effect on the cilia beat frequency of ciliated tissue taken from the trachea of a chick embryo according to the model that is well known in the art (“The Effect of Nasal Drug Formulations on Ciliary Beating in Vitro,” Romeijn, et al.; Int. J. Pharm., 135; 1996: 137-145; “Classification of Cilio-Inhibiting Effects of Nasal Drugs,” Merkus, et al., Laryngoscope, 111(4); April 2001: 595-602). By “ameliorating said toxicity” herein is meant that the adverse effect of the active agent on the cilia beat frequency is lessened as a result of the inclusion in the medicament of ascorbic acid for co-administration with said active agent. Such lessening of the adverse effect of the active agent on the cilia beat frequency may comprehend lessening the reduction in cilia beat frequency or allowing the cilia beat frequency to recover, at least to an extent, upon removal of the active agent, for example by washing, even after repeated administration of the active agent.

In some embodiments, the active agent may be “toxic” to ciliated tissue to the extent that upon administration to such tissue it reduces the cilia beat frequency to 0% or substantially 0% (i.e., less than 5% and, in some instances, less than 1%). The “toxic” effect of the active agent may also be irreversible, in the sense that upon removal of the active agent, for example by washing, the cilia beat frequency does not recover or does not recover significantly (i.e., after washing, the cilia beat frequency may remain at less than 10% and, in some instances, less than 5%).

Further particulars of the cilia beat frequency model are given below.

In some embodiments, said active agent may comprise apomorphine, optionally in the form of a pharmaceutically acceptable salt or hydrate, e.g., apomorphine HCl. Suitably, said medicament may comprise 0.1-50% wt. apomorphine, preferably 1-15% wt., and typically 1-10% wt. The medicament of the present invention may be used in the treatment of Parkinson's disease or other diseases, disorders or symptoms for which apomorphine is indicated such, for example, as sexual dysfunction.

Said medicament is formulated as a powder for nasal administration, and may comprise one or more excipients that are appropriate for such formulation and are known to those skilled in the art. In some embodiments, said medicament may comprise about 99.8-45% wt. of such one or more excipients in addition to said apomorphine and said ascorbic acid, the total amount of ingredients being 100% wt. Preferably, said medicament comprises about 70% wt. or more of said one or more excipients, typically about 80% wt. or more.

Typically, said powder may have a mean particle size in the range from 5-150 μm, preferably about 50-100 μm. Further, the amounts of particles with a size less than 5 μm or greater than 150 μm should desirably be minimised.

Advantageously, said medicament may be freeze-dried. Suitably, said medicament may comprise one or more sugar alcohols as a freeze-drying excipient. Known sugar alcohols comprise arabitol, erythritol, glycerol, isomalt, lactitol, maltitol, mannitol, sorbitol and xylitol. Preferably, said one or more sugar alcohols may be selected from mannitol, sorbitol; inositol and/or xylitol. Preferably, said sugar alcohol comprises mannitol. In some embodiments, mannitol may be the sole sugar alcohol excipient, such that the medicament consists essentially of apomorphine, ascorbic acid and mannitol.

Suitably, said medicament may comprise about 99.8-40% wt. sugar alcohol, preferably 99.8-73% wt., and typically 99.8-88% wt. In some embodiments, therefore, said sugar alcohol(s) may constitute all or substantially all of the one or more excipients included in the medicament apart from the ascorbic acid.

Alternatively, said medicament may comprise a disaccharide as an additional excipient. Suitably, said medicament may therefore comprise 0-5% wt. of a suitable disaccharide such, for example, as trehalose or sucrose. In some embodiments, said medicament may comprise 0.1-1% or 2% wt. trehalose or sucrose.

Desirably, said medicament should comprise less than about 1% wt. H₂O.

As described in WO-A-2004/075824, by the selection of components with a specific crystalline/amorphous balance, a powdered medicament having a particle size that is suitable for nasal delivery may be directly obtained by freeze-drying, without the need for post-drying milling, and without containing pronounced finings. The freeze-dried medicament according to the present invention therefore preferably comprises an amorphous component, which amorphous component constitutes at least 0.1% wt. or at least 0.5% wt. of the total medicament. In some embodiments, 0.1-15% wt. of the blend may be in an amorphous state, preferably 1-10% wt. In some embodiments, substantially all of the excipient(s), e.g., mannitol, as well as the ascorbic acid may be in crystalline form, whilst substantially all of the active agent, e.g., apomorphine, may be in amorphous form.

The ratio and persistence of the amorphous and crystalline components of the medicament according to the invention may be determined for compliance with the crystalline/amorphous parameters as defined above by thermal analysis, including differential scanning calorimetry.

The particle size distribution pattern of the medicament may be defined by particle size characterisation by laser diffraction.

In some embodiments, such particle size characterisation may be carried out directly on a dry powder sample of the medicament (dry analysis) or on a sample of the medicament suspended in a solvent in which the medicament is insoluble (wet analysis) using, e.g., Mastersizer™ instrumentation available from Malvern Instruments UK. Each sample may be fully de-aggregated at the time of characterisation, and this is best achieved using the wet analysis method. With such method, de-aggregation of particle agglomerates may be achieved by the use of dispersing agents, surfactants and/or sonication of the sample prior to analysis and maintained by stirring or recirculation of the sample during analysis. In addition, de-aggregation of the sample can be verified visually under a microscope.

Alternatively, particle size characterisation may be performed on a sample of the medicament that is aspirated into a detector at a flow rate that approximates how the medicament would be administered by a patient in use, i.e., a flow rate in the range of about 10-30 L/min, typically 15-20 L/min. For this, particle size distribution may be defined using, e.g., instrumentation available from Sympatec GmbH, Germany, with the sample being delivered to the detector using an INHALER™ module that is adapted to simulate the particle size distribution of a sample when administered nasally by a patient. In such case, it is an unnecessary fully to de-aggregate the sample before characterisation, as the intention is to simulate how the medicament would actually be dispensed in practice.

By complying with the crystalline/amorphous parameters defined above, and provided that an aqueous formulation of the components is compatible with freeze-drying, then the medicament according to the invention preferably has one or more of the following properties:
A particle size suitable for nasal delivery that can be induced and maintained by freeze-drying;
A small particle size distribution wherein the proportion of small particles having a size less than about 5 μm, (finings) is minimised;
When removed from the freeze-dryer and exposed to the atmosphere, the particles of the medicament do not alter in size nor absorb moisture to the extent that the particles agglomerate or become sticky, thereby preventing final finishing or dispensing and also influencing pharmacological activity;
The resultant nasal powder exhibits high solubility, improved nasal absorption and, as a consequence, high pharmacological activity.

More particularly, the particle size, as measured by laser diffraction, under which 10% by volume of the particles is distributed ((D(v, 0.1)) is at least 5 μm, preferably at least 6 μm, more preferably at least 9 μm, most preferably at least 10 μm, and particularly preferably at least 15 μm.

The particle size, as measured by laser diffraction, under which 90% by volume of the particles is distributed (D(v, 0.9)) is preferably at most 150 μm, more preferably at most 125 μm, most preferably at most 100 μm, particularly preferably at most 18 μm, and more particularly preferably 50 μm, especially 45 μm.

The particle size distribution (calculated as the difference between (D(v, 0.9) and (D(v, 0.1) is preferably at most 140 μm, more preferably at most 110 μm, most preferably 50 μm, and particularly preferably 40 μm.

The present inventors have observed that the addition of ascorbic acid to a medicament comprising an active agent which exhibits toxicity to ciliated tissue such, for example, as apomorphine surprisingly ameliorates such toxicity to the extent that such tissue taken from the trachea of a chick embryo when treated with said active agent and ascorbic acid recovers cilia beat movement after washing. Furthermore, it has been demonstrated that such tissue retains the ability to recover cilia beat movement even after repeated administration of said active agent and ascorbic acid.

By “ameliorates such toxicity” herein is meant that upon the administration of the active agent in combination with ascorbic acid to ciliated tissue, a cilia beat frequency of at least about 10% of the pre-treatment frequency and preferably at least about 20%, e.g., 20-40% is retained. Further, after washing so as to remove the active agent substantially from the ciliated tissue, the cilia beat frequency recovers to least about 50% of its pre-treatment level, preferably at least about 60% or 75% e.g., at least 80%. In some embodiments, the cilia beat frequency may recover to least about 40-50% of its pre-treatment level within about 20-80 minutes after washing.

In accordance with the present invention, the medicament comprises an amount of ascorbic acid, which amount is at least an amount effective for ameliorating said toxicity. Said medicament may comprise up to about 5% wt. ascorbic acid. Suitably, the medicament of the present invention may comprise about 0.1-5% wt. ascorbic acid, preferably 0.5-2% wt., e.g., about 1% wt.

The medicament according to the invention may be manufactured by freeze-drying a solution comprising said active agent, a sugar alcohol and ascorbic acid, so that least 0.1% wt. of the freeze-dried blend is in an amorphous state.

Suitably, said solution may therefore comprise an admixture of 0.1-50% wt. of said active agent, 0.1-5% wt. of ascorbic acid, 0-5% wt. trehalose and/or sucrose and 99.8-40% wt. of said sugar alcohol.

As described in WO-A-2004/075824, the freezing conditions should preferably be selected to provide an optimal ice crystal structure conducive to maximal sublimation rate, the maintenance of the crystalline phase within the matrix, and/or induction of and/or maintenance of an amorphous phase within the matrix.

The selection of suitable freezing conditions will be influenced by the chemical nature and concentration of the active component(s) and crystallising or amorphous excipient(s) within the solution or suspension, freeze-dryer design and specification, the primary container used to process the product and/or sample fill depth.

Differential scanning calorimetry, differential thermal analysis and resistance analysis may be used to define optimum freezing conditions. From such analysis, it has been found that it is desirable that the product should be frozen at a slow rate or heat annealing cycle applied to induce or maintain the correct matrix composition. For example, a freezing rate of about 0.1 to 0.5° C. per minute and heat annealing cycle comprising, for example: cool products to −45° C. at 0.1-1.0° C. per minute; hold 2 hours, warm to −15° C., hold 2 hours, re-cool to −45° C., hold 2 hours before drying, have been used. These values may be used for guidance, but will vary depending on the formulation of the active material and limitations introduced by the apparatus and other component(s) used in freeze-drying.

For example, a suitable drying cycle includes heating directly to 5° C. for main drying, increased chamber pressure to 150 mTorr to facilitate heat input and increased final drying temperature to 20° C. Variations on this cycle, designed for specific product/process optimisation included cycle where shelf temperature is raised to 15° C. for the initial phase of main (primary) drying and then progressively reduced to 5° C. for the remainder of main (primary) drying with chamber pressure increased up to 300 mTorr to facilitate heat input into product followed by increased shelf temperature to 25° C. for final (secondary) drying.

Factors which determine the freeze-drying characteristics of the sample include:
The glass transition temperature (Tg′) which determines the temperature at which the viscosity of the cooled mass decreases sufficiently so that the sample collapses during freeze-drying. Glass transition temperatures have been determined by differential scanning calorimetry or differential thermal analysis;
Operationally the temperature at which the sample collapses during freeze-drying is defined as the collapse temperature (Tc). Collapse temperatures are determined by freeze-drying microscopy. In the absence of complicating factors such as the development of surface skins on the drying sample, collapse and glass transition temperatures are typically similar;
Skin formation and associated defects, are also determined by freeze-drying microscopy.

The medicament according to the invention has the advantage that no preservatives such, for example, as bactericides and fungicides, are necessary. Such preservatives are known to decrease the ciliary movement (Romeijn, et al., 1996, Merkus, et al., 2001).

The medicament according to the invention may be administered using a nasal insufflator or a passive device. In some embodiments, the medicament may, for example, be placed in a capsule which is disposed in an inhalation or insufflation device. A needle may be penetrated through the capsule to make pores at a top and bottom of the capsule and air may be drawn in by inhalation or blown through the device to force out the particles of medicament into the patient's nose. The medicament may also be administered in a jet-spray of an inert gas or suspended in liquid organic fluids. The required amount for nasal administration of a nasal medicament according to the invention may be, for example, between 1 and 50 mg, typically 1 to 20 mg, for example administered as about 5 to 20 mg per nostril.

In some embodiments, medicament may comprises:

| | w/w |
|---|---|
| Apomorphine | 0.1-50% |
| Mannitol | 99.8-45% |
| Ascorbic acid | 0.1-5%. |

Alternatively, said medicament may comprise:

| | w/w |
|---|---|
| Apomorphine | 0.1-15% |
| Mannitol | 99.8-80% |
| Ascorbic acid | 0.1-5%. |

Alternatively, said medicament may comprise:

| | w/w |
|---|---|
| Apomorphine | 0.1-10% |
| Mannitol | 99.8-88% |
| Ascorbic acid | 0.1-2%. |

Following is a description by way of example only with reference to the accompanying drawings of embodiments of the present invention.

In the drawings:

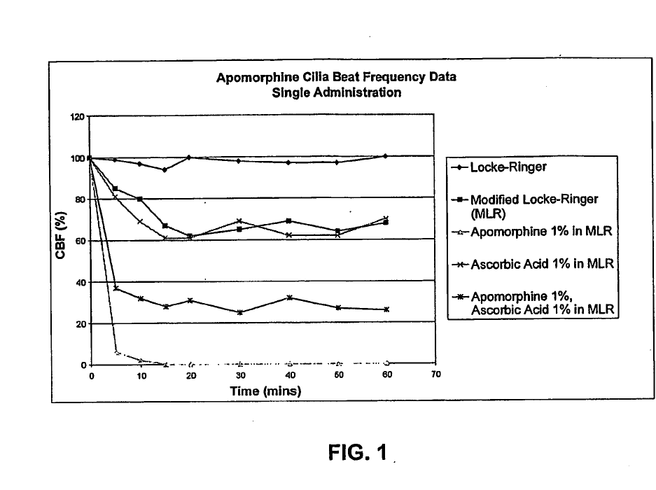

FIG. 1 is a graph of cilia beat frequency versus time following respective single administrations of various substances to ciliated tissue taken from the trachea of a chick embryo to show the relative effects of such substances;

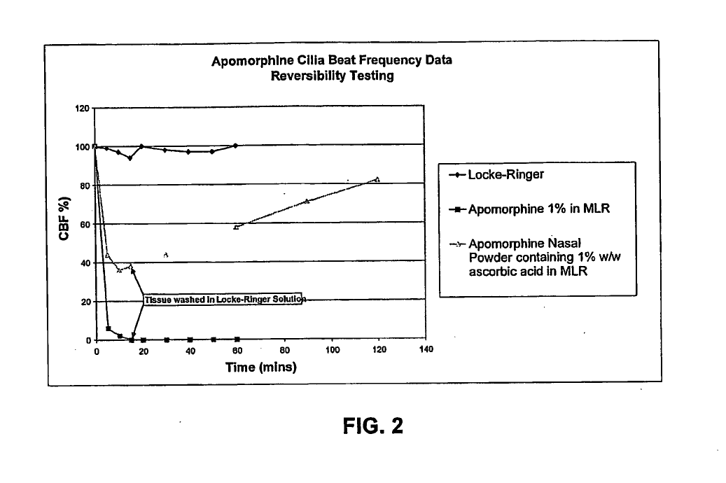

FIG. 2 is a graph of cilia beat frequency versus time following respective single administrations of various test solutions to ciliated tissue taken from the trachea of a chick embryo followed by washing such tissue with the Locke-Ringer solution after 15 minutes exposure to the solutions;

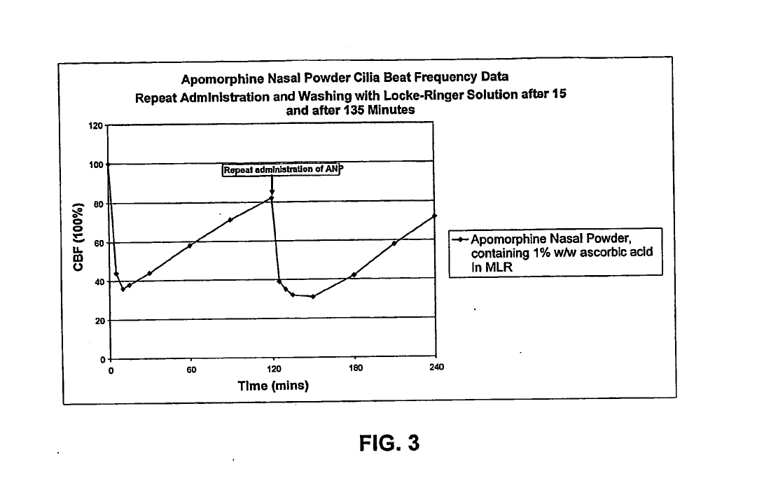

FIG. 3 is a graph of cilia beat frequency versus time following repeated administration of apomorphine nasal powder in a solution containing 1% ascorbic acid and subsequent washing with Locke-Ringer solution.

### EXAMPLE 1

A powered medicament comprising 2.5% w/w apomorphine according to the present invention was formulated with the following composition:

| | w/w |
|---|---|
| Apomorphine HCl | 2.5% |
| Mannitol | 96.5% |
| Ascorbic acid | 1% |

The medicament was manufactured by dissolving the above-recited constituents in water to form a solution, and thereafter freeze-drying the solution to form the powdered medicament. The following freeze-drying cycle was used:

Freeze to −45° C.

Cooling rate 0.25° C. per minute

Hold 120 minutes

Main Drying:

Shelf Temperature (step 1)-20° C.

Warming rate 1.0° C. per minute

Hold 1200 minutes

Chamber pressure 50 mTorr

Shelf Temperature (step 2) 0° C.

Warming rate 1.0° C. per minute

Hold 720 minutes

Chamber pressure 50 mTorr

Shelf Temperature (step 3) 5° C.

Warming rate 1.0° C. per minute

Hold 1000 minutes

Chamber pressure 50 mTorr

Final Drying

Shelf Temperature 15° C.

Warming rate 1.0° C. per minute

Hold 700 minutes

Chamber pressure 50 mTorr

The above-freeze-drying cycle has been used effectively for formulations having Tg′ or Tc at about −16 to −18° C. This means that the product temperature should be maintained at about −23° C. (i.e., −18° C. minus 5° C. for operational safety=−23° C.)

The particle size distribution of the powdered medicament was measured by using a Malvern Instruments Mastersizer™ laser diffraction machine having a 300 mm range lens, a beam length of 14.30 mm and sampler MS7. Hexane was used as a solvent, the wetter was Span 85 and the medicament was sonicated for 1 minute before being measured. The results of the particle size distribution measurements are given in terms of the particle size at which 10%, 50% and 90% by volume of the particles exist which are referred to as D(v, 0.1), D(v, 0.5) and D(v, 0.9). The size difference between D(v, 0.9) and D(v, 0.1) has also been calculated, as this indicates the particle size range. In other words, the lower the size difference, the narrower the particle size distribution curve and the less poly-disperse the distribution.

The medicament manufactured as described above was found to have D(v, 0.1)=6.52 μm, D(v, 0.5)=22.40 μm, D(v, 0.9)=55.12 μm and D(v, 0.9)−D(v, 0.1)=48.60 μm.

### EXAMPLES 2-7

The further medicaments according to the present invention listed in Table 1 below were prepared in accordance with the method as described in Example 1 above. The corresponding particle size measurements are also given in the Table.

The dissolution data for Example 6 is given in WO-A-2004/075824 (pages 21-22 and FIG. 1).

The pharmacokinetic (PK) data for Examples 2 and 7 above are given in WO-A-2004/075824 (pages 21-23 and FIG. 3), demonstrating that the nasal administration route is viable with the medicaments according to the present invention.

WO-A-2004/075824 (pages 24-26) demonstrates the rapid efficacy of a medicament according to Example 5 above in reversing an “off” period in subjects with Parkinson's disease known to respond to a single dose of ≦5 mg subcutaneous apomorphine.

TABLE 1

| Example No. | Formulation | D(v, 0.1)/μm | D(v, 0.5)/μm | D(v, 0.9)/μm | D(v,0.9) − D(v,0.1)/μm |
|---|---|---|---|---|---|
| 2 | Apomorphine 10% w/w | 15.42 | 55.88 | 124.24 | 108.82 |
| | Mannitol 89% w/w | | | | |
| | Ascorbic Acid 1% w/w | | | | |
| 3 | Apomorphine 10% w/w | 5.72 | 19.05 | 40.31 | 34.59 |
| | Mannitol 88.9% w/w | | | | |
| | Ascorbic Acid 1% w/w | | | | |
| | Trelahose 0.1% w/w | | | | |
| 4 | Apomorphine 10% w/w | 6.01 | 19.76 | 40.52 | 34.51 |
| | Mannitol 88% w/w | | | | |
| | Ascorbic Acid 1% w/w | | | | |
| | Trelahose 1% w/w | | | | |
| 5 | Apomorphine 25% w/w | 10.13 | 28.62 | 54.35 | 44.22 |
| | Mannitol 74% w/w | | | | |
| | Ascorbic Acid 1% w/w | | | | |
| 6 | Apomorphine 50% w/w | 5.79 | 20.61 | 41.85 | 36.06 |
| | Mannitol 49% w/w | | | | |
| | Ascorbic Acid 1% w/w | | | | |
| 7 | Apomorphine 2.5% w/w | | | | |
| | Ascorbic Acid 1% w/w | | | | |
| | Trelahose 1% w/w | | | | |
| | Mannitol 95.5% w/w | | | | |
| 8 | Apomorphine 10% w/w | | | | |
| | Sucrose 1% w/w | | | | |
| | Inositol 88% w/w | | | | |
| | Ascorbic Acid 1% w/w | | | | |

In this Table Apomorphine means the HCl salt

### EXAMPLE 8

The cilia beat frequency (CBF) model is a recognised technique to investigate the relative toxicity of substances applied in solution to a section of ciliated tissue taken from the trachea of a chick embryo (Romeijn, et al., 1996, Merkus, et al., 2001). The tissue is maintained in Locke-Ringer solution and viewed under a microscope to assess the rate at which the cilia beat. The tissue is then transferred to a well containing 1.0 ml of test solution and the impact on CBF assessed. Further, the reversibility of any effects can be measured by washing the tissue in Locke-Ringer solution 15 minute post administration and evaluating the recovery of CBF.

Single Administrations

FIG. 1 shows the relative effects of a single administration of various solutions in the CBF model. The toxicity of apomorphine is seen by the complete arrest of cilia beat movement induced by the exposure of the tissue to a ‘1% apomorphine in Modified Locke-Ringer (MLR)’ solution. This effect is ameliorated significantly by the addition of 1% wt. ascorbic acid to this solution.

Reversibility Assessment

FIG. 2 shows the effect of washing the tissue in Locke-Ringer solution 15 minutes after separate administrations of ‘1% apomorphine in MLR’ solution and ‘Apomorphine 25% w/w Nasal Powder (ANP) 1% in MLR’ solution containing 1% wt. ascorbic acid.

Apomorphine 25% w/w Nasal Powder comprises 25% w/w apomorphine HCl, 1% w/w ascorbic acid and 74% w/w mannitol. In this experiment an amount of 400 mg of Apomorphine 25% Nasal Powder was dissolved in 10 ml of MLR to a concentration of 1.0% w/v apomorphine. It is clear that the toxic effects of apomorphine alone are irreversible while the tissue treated with the ANP formulation containing 1% w/w ascorbic acid recovers after washing with Locke-Ringer.

Repeat Administration

FIG. 3 shows the effect of a repeat administration and washing with Locke-Ringer solution of Apomorphine 25% w/w Nasal Powder 1% in MLR containing 1% w/w ascorbic acid prepared as described above. It is clear that the viability of the tissue is unaffected with the effect and recovery of each administration being similar.

1-9. (canceled)

10. A method for ameliorating the toxicity to ciliated tissue of a nasally-administered powdered medicament, the method comprising administering the medicament with ascorbic acid, wherein the medicament comprises an active agent that is toxic to ciliated tissue.

11. The method of claim 10, wherein said active agent comprises apomorphine, optionally in the form of a pharmaceutically acceptable salt or hydrate.

12. The method of claim 10, wherein said medicament is freeze-dried.

13. The method of claim 12, wherein said medicament comprises at least one sugar alcohol as a freeze-drying excipient.

14. The method of claim 12, wherein said freeze-dried medicament comprises an amorphous component, which amorphous component constitutes at least 0.5% wt. of the total medicament.

15. The method of claim 10, wherein said medicament comprises 0.1-5% wt. ascorbic acid.

16. The method of claim 10, wherein said medicament comprises 0.1-50% wt. apomorphine.

17. The method of claim 10, wherein said medicament comprises 99.8-45% wt. mannitol.

18. The method of claim 13, wherein the at least one sugar alcohol comprises mannitol.
